(19) United States
(12) Patent Application Publication
(10) Pub. No.: US 2006/0275217 A1
(43) Pub. Date: Dec. 7, 2006

(54) Chemical exchange saturation transfer contrast agents

(76) Inventors: Peter D. Caravan, Cambridge, MA (US); Vincent Jacques, Somerville, MA (US); Randall B. Lauffer, Brookline, MA (US); Heribert Schmitt-Willich, Berlin (DE)

Correspondence Address: FISH & RICHARDSON P.C., PO BOX 1022, MINNEAPOLIS, MN, 55440-1022, US

(21) Appl. No.: 11/429,338

(22) Filed: May 5, 2006

Related U.S. Application Data
(60) Provisional application No. 60/678,613, filed on May 6, 2005.

(51) Int. Cl. A61K 49/10; C07F 5/00

(52) U.S. Cl. 424/9.363; 534/16

(57) ABSTRACT

Chelating ligands and metal chelates useful as CEST MR contrast agents are disclosed. The CEST agents can be used to evaluate blood volume changes in the heart and brain.

### CROSS-REFERENCE TO RELATED APPLICATION

This application claims priority under 35 U.S.C. § 119 to U.S. Provisional Application Ser. No. 60/678,613, filed May 6, 2005, incorporated by reference herein.

### TECHNICAL FIELD

This invention relates to magnetic resonance imaging (MRI) contrast agents, and in particular, to MR contrast agents that are useful as chemical exchange saturation transfer (CEST) contrast agents. Methods of using CEST agents for assessing blood volume changes in the heart and brain are also disclosed, as well as methods for imaging microthrombi in the brain.

### BACKGROUND

Ischemic heart disease is a leading cause of death in the developed world. Efforts in the detection of the disease often focus on the patency of major blood vessels such as the coronary arteries, and recent paradigms have emphasized the importance of the coronary microvasculature in providing blood flow, including collateral blood flow, to injured myocardial tissue. Because ischemically-injured myocardium contains both reversibly and irreversibly injured regions, accurate characterization of myocardial injury, in particular the differentiation between necrotic (acutely infarcted myocardium), ischemic, and viable myocardial tissue, is an important factor in proper patient management. This characterization can be aided by an analysis of the perfusion and/or reperfusion state of myocardial tissue adjacent to coronary microvessels either before or after an ischemic event (e.g., an acute myocardial infarction).

Because cardiac catheterization assessing the patency of coronary arteries is an expensive and risky procedure, noninvasive techniques that assess the likelihood of coronary artery disease have flourished. Myocardial perfusion may be assessed using several diagnostic techniques that use a stress/rest paradigm (see Marcus Cardiac Imaging, 2^{nd} ed., D. J. Skorton, H. R. Schelbert, G. L. Wolf, and B. H. Brundage, eds, W. B. Saunders, Philadelphia, 1996). Here, some measure of blood flow is determined at rest, and then the measurement is repeated when blood flow is increased because of either exercise or pharmacologic stress. The difference between the two images provides a relative measure of perfusion. Myocardial perfusion measurements rely on the fact that myocardial blood flow increases going from a resting state to a state of hyperemia.

Recently, magnetic resonance imaging (MRI) techniques have also been proposed to assess myocardial perfusion. In general, MRI is appealing because of its noninvasive character, ability to provide improved spatial resolution, and ability to derive other important measures of cardiac performance, including wall motion and ejection fraction in a single sitting. Many MRI perfusion imaging techniques require rapid imaging of the myocardium during the first pass (after bolus injection) of an extracellular or intravascular MR contrast agent; this technique is referred to as MRFP (magnetic resonance first pass) perfusion imaging. On Ti-weighted images, the ischemic zones appear with a delayed and lower signal enhancement (e.g., hypointensity) as compared with normally perfused myocardium. Myocardial signal intensity versus time curves can then be analyzed to extract perfusion parameters. Intensity differences, however, rapidly decrease as the MR contrast agent is diluted in the systemic circulation after the first pass. Furthermore, because of the rapid timing requirement of MRFP perfusion imaging, the patient must undergo pharmacologically-induced stress while positioned inside the MRI apparatus, and rapid imaging may limit the resolution of the perfusion maps obtained, resulting in poor quantification of perfusion. In addition, two injections of contrast are required and two sets of serial images must be examined.

Another organ where changes in blood flow are studied is the brain, e.g., to assess ischemia or for functional brain imaging. Functional MRI (fMRI) is a technique that measures changes in blood flow in the brain during specific activation, i.e. when the subject is subjected to visual or auditory stimuli. Here differences in blood oxygen levels during rest and stress give rise to signal differences that can be quantified. However, the fMRI technique may not be as sensitive as is necessary for useful studies. PET can also be used for functional brain studies, where 015 labeled water is used as a tracer of blood flow. The main drawback with this technique, however, is the very short half-life of the tracer, limiting where studies can be performed.

Microthrombosis (thrombi in the capillaries of the brain) is implicated in many diseases such as ischemic cerebral infarction. It is difficult to detect and quantify microthrombi because of the very small volume that the capillaries occupy in the brain.

Traditional MR contrast agents are limited in the diagnosis of microthrombosis because hey alter the relaxation properties of only a small volume of water, making detection difficult.

Many of the metal chelating ligands currently used in MR are polyaminopolycarboxylate metal binding chelating ligands derived from two basic structures, DOTA and DTPA. These ligands are used typically because of their known affinity for metal ions, including gadolinium(III). Researchers have recently described certain lanthanide complexes that consist of a trivalent lanthanide (e.g., Eu, Th, Dy, Ho, Er, Tm, or Yb), a coordinated water ligand, and a tetraamide-cyclen octadentate ligand that can be used as CEST-type contrast agents for MRI. See, e.g., J. Am. Chem. Soc. (2001) vol. 123:1517 and Angew. Chemie, Intl. Ed. Engl. (2002) 41: 4334. CEST agents function by having exchangeable hydrogen atoms (e.g., the coordinated water in the references outlined above) resonating at a different frequency (ω₁) than water. When a radiofrequency (rf) pulse is applied at the frequency of the exchangeable hydrogen (i.e., a saturation pulse), some of the magnetization (saturation) is transferred to the bulk water hydrogens. The result of this magnetization exchange is a decrease in magnetization (and signal) for bulk water where the CEST agent is present. The effect is only observed when the rf pulse is applied at the frequency of the exchangeable hydrogen.

In CEST imaging, two images are acquired and combined to create a third CEST specific image. For example, one image is acquired with selective irradiation at the exchangeable hydrogen frequency (Δω=frequency difference between the exchangeable hydrogen resonance and the water resonance), and another image is acquired with irradiation at a different frequency (e.g., a frequency equal to but opposite that of the first (−Δω)) to minimize effects of macromolecular interference, T2, T1, and in-homogeneity artifacts. The two images are then combined (e.g., by subtraction or division) to create a third image that is characteristic of the CEST agent.

In order to obtain the maximum CEST effect and hence to provide greater image contrast (or to effect contrast at lower concentration of contrast agent), the rate of exchange of the bound water should be optimized. The water exchange rate should be as fast as possible but still meet the so-called “slow exchange limit” ωτ>1, where ω is the chemical shift difference between the bound water and the bulk water resonances and τ is the residency time of the bound water (the inverse of the exchange rate). For a given chemical shift difference, there is an optimal exchange rate. It would be useful, therefore, to have CEST agents that combine a large chemical shift difference with a fast water exchange rate that can be used to assess perfusion and blood volume changes in the heart and brain.

### SUMMARY

The invention is based on the discovery that modifications of donor groups on a chelating ligand can yield a resultant metal chelate that is useful as a CEST contrast agent. In certain cases, donor groups may be able to coordinate a metal ion. In other cases, donor groups allow the contrast agent to bind to particular physiologic targets in vivo. The donor groups can include a number of functionalities to exploit CEST mechanisms, including, by way of example, enhancing the water exchange rate of one or more protons, or increasing the number of exchangeable protons.

In addition, CEST agents described herein provide a novel mechanism for monitoring perfusion in, e.g., the heart and brain. While other perfusion techniques rely on a difference in blood flow to assess perfusion, the present invention takes advantage of the increase in blood volume in tissues during stress, e.g., hyperemia. For instance, during hyperemia in the heart, the blood volume in the myocardium increases by a factor of two during full vasodilatory stress. Similarly, blood volume in the gray matter of the brain increases 15%-30% under specific activation. Determining changes in blood volume therefore provides a surrogate measurement of perfusion to blood flow. By comparing blood volume at stress and rest, the present CEST agents make it possible to identify ischemic areas in, for example, the heart and brain. The CEST agents are also useful for detecting microthrombi in the brain.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the methods, materials, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1. ₁H NMR (top) and CEST (bottom) spectra in H₂O/D₂O of the Eu-polylysine derivative described in Example 4a.

FIG. 2. Digital difference axial image between control image with saturation centered at −52 ppm (−10400 Hz) from the water resonance and CEST image with saturation centered at +52 ppm (10400 Hz), showing contrast enhancement in the blood pool. The Eu-polylysine derivative described in Example 4a was used to generate the images.

FIG. 3. Reference images that were subtracted digitally to give FIG. 2. The image on the left (FIG. 3A) is the reference with saturation at −52 ppm; the one on the right (FIG. 3B) is with saturation at +52 ppm.

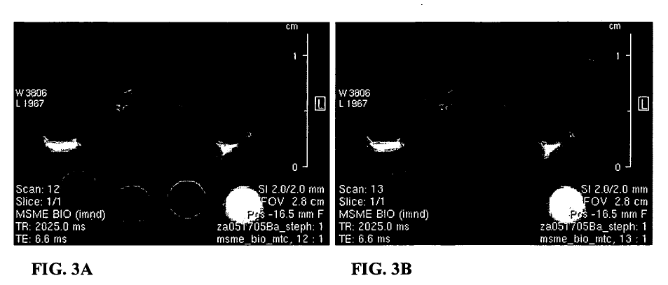

### DETAILED DESCRIPTION

Definitions

Commonly used chemical abbreviations that are not explicitly defined in this disclosure may be found in The American Chemical Society Style Guide, Second Edition; American Chemical Society, Washington, DC (1997), “2001 Guidelines for Authors” J. Org. Chem. 66(1), 24A (2001), “A Short Guide to Abbreviations and Their Use in Peptide Science” J. Peptide. Sci. 5, 465-471 (1999).

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. Moreover, the term “alkyl” includes both “unsubstituted alkyls” and “substituted alkyls,” the latter of which refers to alkyl moieties having substituents replacing a hydrogen on one or more carbons of the hydrocarbon backbone. An “arylalkyl” moiety is an alkyl substituted with an aryl (e.g., phenylmethyl (benzyl)). An alkyl group can contain from about 2 to about carbon atoms, e.g., 2, 3, 4, 5, 6, 7, 8, 9, 10, 11, or 12 C atoms.

In general, the term “aryl” includes groups, including 5- and 6-membered single-ring aromatic groups that may include from zero to four heteroatoms, for example, benzene, phenyl, pyrrole, furan, thiophene, thiazole, isothiaozole, imidazole, triazole, tetrazole, pyrazole, oxazole, isooxazole, pyridine, pyrazine, pyridazine, and pyrimidine, and the like. Furthermore, the term “aryl” includes multicyclic aryl groups, e.g., tricyclic, bicyclic, such as naphthalene, benzoxazole, benzodioxazole, benzothiazole, benzoimidazole, benzothiophene, methylenedioxyphenyl, quinoline, isoquinoline, napthridine, indole, benzofuran, purine, benzofuran, deazapurine, or indolizine. Those aryl groups having heteroatoms in the ring structure may also be referred to as “aryl heterocycles,” “heterocycles,” “heteroaryls,” or “heteroaromatics.” An aryl group may be substituted at one or more ring positions with substituents.

The terms “chelating ligand,” “chelating moiety,” and “chelate moiety” may be used to refer to a polydentate ligand which is capable of coordinating a metal ion, either directly or after removal of protecting groups, or is a reagent, with or without suitable protecting groups, that is used in the synthesis of a contrast agent and comprises substantially all of the atoms that ultimately will coordinate the metal ion of the final metal complex. The terms “chelate” or “metal chelate” refer to the actual metal-ligand complex, and it is understood that the polydentate ligand can eventually be coordinated to a medically useful or diagnostic metal ion.

As used herein, the term “purified” refers to a peptide that has been separated from either naturally occurring organic molecules with which it normally associates or, for a chemically-synthesized peptide, separated from any other organic molecules present in the chemical synthesis. Typically, the polypeptide is considered “purified” when it is at least 70% (e.g., 70%, 80%, 90%, 95%, or 99%), by dry weight, free from any other proteins or organic molecules.

As used herein, the term “peptide” refers to a chain of amino acids that is about 2 to about 75 amino acids in length (e.g., 3 to 50 amino acids, or 3 to 30 amino acids). All peptide sequences herein are written from the N to C terminus. Additionally, peptides containing two or more cysteine residues can form disulfide bonds under non-reducing conditions.

As used herein, the term “natural” or “naturally occurring” amino acid refers to one of the twenty most common occurring amino acids. Natural amino acids modified to provide a label for detection purposes (e.g., radioactive labels, optical labels, or dyes) are considered to be natural amino acids. Natural amino acids are referred to by their standard one- or three-letter abbreviations.

The term “non-natural amino acid” or “non-natural” refers to any derivative of a natural amino acid including D forms, and β and γ amino acid derivatives. It is noted that certain amino acids, e.g., hydroxyproline, that are classified as a non-natural amino acid herein, may be found in nature within a certain organism or a particular protein.

The term “specific binding affinity” as used herein, refers to the capacity of a contrast agent to be taken up by, retained by, or bound to a particular biological component to a greater degree than other components. Contrast agents that have this property are said to be “targeted” to the “target” component. Contrast agents that lack this property are said to be “non-specific” or “non-targeted” agents. The binding affinity of a binding group for a target is expressed in terms of the equilibrium dissociation constant “Kd.”

The terms “target binding” and “binding” for purposes herein refer to non-covalent interactions of a contrast agent with a target. These non-covalent interactions are independent from one another and may be, inter alia, hydrophobic, hydrophilic, dipole-dipole, pi-stacking, hydrogen bonding, electrostatic associations, or Lewis acid-base interactions.

Design of Chelating Ligands

The invention relates to chelating ligands useful for preparing CEST metal chelates. CEST metal chelates can be used as MR agents, which can be referred to herein as “CEST agents” or “CEST contrast agents.” CEST chelating ligands coordinate lanthanide ions to yield CEST metal chelates. Suitable lanthanides include: Pr(III), Nd(III), Eu(III), Tb(III), Dy(III), Er(III), Ho(III), Tm(III), Ce(III), and Yb(III). In addition, the CEST chelating ligands and metal chelates can include target binding moieties (TBMs) and/or Linker moieties (Ls). Chelating ligands having target binding moieties allow the chelating ligands (and CEST metal chelates) to be targeted to various sites in vivo. Both monomeric and multimeric CEST chelating ligands and chelates are provided.

Monomeric CEST Chelating Ligands and Metal Chelates

Monomeric chelating ligands described herein are based on derivatives of a diethyltriamine scaffold or a 1,4,7,10-tetraazacyclodecane scaffold. Derivatives are prepared by including one or more donor groups (D's) on a scaffold, e.g., one, two, three, four, fix, six, or more. In some cases, a D can coordinate a metal ion; in other cases, a D can include a targeting binding moiety and/or a linker. Ds can be chosen to for their ability to enhance the efficacy of the chelating ligand as a CEST agent. CEST efficacy may be enhanced, for example, by enhancing the water exchange rate of one or more protons or by increasing the number of exchangeable protons.

A variety of chelating ligands can be prepared according to the present invention. Chelating ligands of the invention can have a general formula as follows:

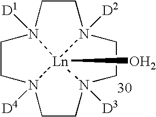

where:

D¹ is any of:

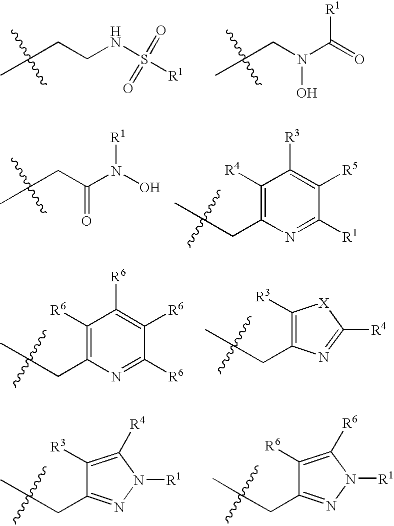

D², D³, D⁴ are any of 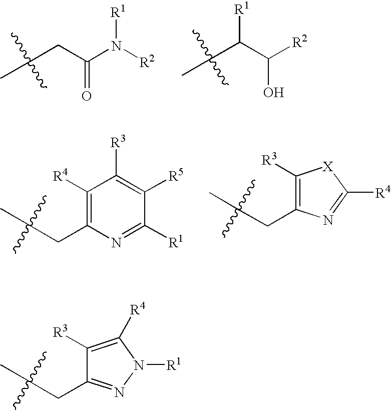
and
R¹=H, 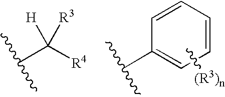
independently 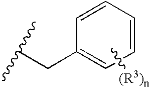
independently
R²=H, 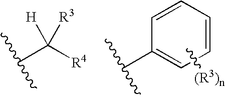
independently 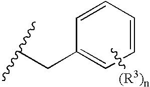
independently
R³=H, alkyl, aryl, benzyl, halogen, carboxylate, sulfonate, or phosphonate;
R⁴=H, alkyl, aryl, benzyl, halogen, carboxylate, sulfonate, or phosphonate;
R⁵=H, alkyl, aryl, benzyl, halogen, carboxylate, sulfonate, or phosphonate;
R⁶=H, R³, R⁴, or together with the atoms to which it is attached forms a substituted or unsubstituted 5 or 6 member aromatic ring or heteroaromatic ring;
X=O, S, N—R¹;
Ln=Eu(III), Nd(III), Pr(III), Ce(III), or Yb(III); and
n=1-5.

Stereochemistries of each D can be independent of one another. Any of the D groups can be modified to couple a targeting group, such as through a -[L]m-[TBM]ₙ moiety, to a chelating ligand. Methods for coupling the D groups to suitable -[L]m-[TBM]n moieties are known to those having ordinary skill in the art. As used herein, each reference to -[L]m-[TBM]ₙ includes the limitation that m can be 0 or 1 and n can range from 1 to 5.

Ds can be chosen based on their effect on various CEST mechanisms. For example, one way to increase CEST efficacy is to take advantage of exchangeable protons, such as amide or hydroxyl protons, in addition to coordinated water molecule protons. In this case, a CEST method can saturate either the proton signals of the exchangeable protons or the proton signals of the coordinated water protons, or both. For example, in a tetra-amide-based CEST agent (e.g., where each D includes an amide moiety), four equivalent amide protons can be exchanged (compared to two for one water molecule). Increasing the acidity of such amide protons by altering the structure of the Ds increases the exchange rate and therefore the efficacy of the CEST agent. Such an effect can be achieved by introducing an electron-withdrawing group (EWG) and/or a H-bond forming group on one or more amide nitrogens. Since water exchange rate is also a function of the nature of the chelated lanthanide ion, proper combinations of D substituents and a lanthanide ion provide a CEST agent with a desirable proton-exchange rate and increased efficacy.

Proton exchanging groups (groups with exchangeable protons such as amide, alcohol and phenol groups) in the second or higher coordination spheres can also be employed to increase CEST efficacy. Extending the system to the second coordination sphere allows for higher numbers of exchangeable protons, which may be identical. An appropriate paramagnetic lanthanide ion generates an induced chemical shift large enough for these protons to give a discrete peak that can be irradiated selectively. One genus of such compounds includes the following structures: 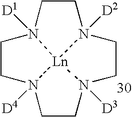
where Ln is Dy(III), Tm(III), Ho(III), Tb(III), Er(III), or Yb(III);
where at least one D², D₂, D⁴ is 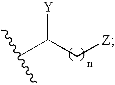
where n=0-4 and Z can be 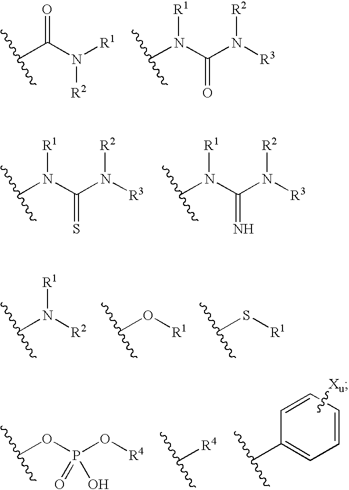
where R¹, R² and R³ are independently H; C((CH₂)ₛ—X)ₜ where t=1-3, s=1-6, and X=NH², OH, SH, CONH₂,NH—CO—NH₂, NH—C(NH)—NH₂, NH—NH₂, aryl-OH, aryl-SH; a sugar residue; CH²-arylX^{u} where X is defined as above and u=1-5; and aryl-X^{u} where X and u are defined as above;
where R⁴ is defined as R¹ but can not be a hydrogen;
where Y is carboxylate, substituted or unsubstituted carboxamide, phosphonate, phosphonate ester, phosphinate, or phosphinate ester;
   and the other D¹, D², D³, D⁴ are 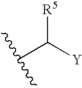
where R⁵ is H, alkyl, cycloalkyl, aryl, benzyl;
and Y is carboxylate, substituted or unsubstituted carboxamide, phosphonate, phosphonate ester, phosphinate, or phosphinate ester.

Another genus of such compounds is as follows: 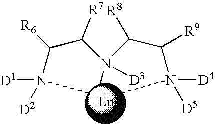
where Ln is Dy(III), Tm(III), Ho(III), Tb(III), Er(III), or Yb(III);
where at least one D¹⁻⁵ is 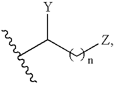
where n=0-4 and Z can be 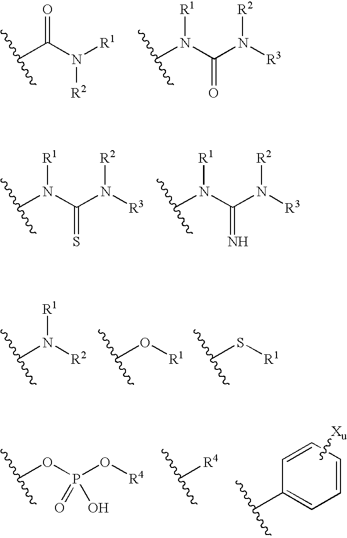
where R₁, R² and R³ are independently H; C((CH₂)^{s}—X)^{t} where t=1-3, s=1-6, and X=NH². OH, SH, CONH₂,NH—CO—NH₂, NH—C(NH)NH₂, NH—NH₂, aryl-OH, aryl-SH; a sugar residue; CH₂-arylX^{u} where X is defined as above and u=1-5; aryl-X^{u} where X and u are defined as above;
where R⁴ is defined as R₁ but can not be a hydrogen;
where Y is carboxylate, substituted or unsubstituted carboxamide, phosphonate, phosphonate ester, phosphinate, or phosphinate ester;
where R⁶, R⁷, R⁸, and R⁹ are defined as R₁ and can also be (CH₂)^{m}—Z
where m=0-6 and Z is defined above;
   and the other D¹⁻⁵ are 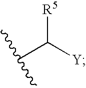
where R⁵ is H, alkyl, cycloalkyl, aryl, benzyl and Y is carboxylate, substituted or unsubstituted carboxamide, phosphonate, phosphonate ester, phosphinate, or phosphinate ester.

Another genus of such compounds includes polymeric derivatives as follows:
C-L)^{s}-P-(L′-E)^{t}
where C is a contrast agent as defined in the two classes immediately above;
where E is selected from NH2, OH, SH, CONH2,NH—CO—NH2, CO—NR1R2, NH—C(NH)—NH2, NH—NH2, aryl-OH, and aryl-SH;
where R₁ and R2 are as defined in the two classes immediately above;
where L and L′ are linkers, e.g., as described herein;
where s=1-100 and t=0-100; and
where P is a polymer or dendrimer.

P can be a positively charged polymer or dendrimer, e.g., a polymer or dendrimer having multiple amino groups. In some embodiments, the (C-L) moieties can be bound through one or more of the amino groups of P. In cases where one or more the amino groups are not so bound, one or more of the free amino groups can be capped, e.g., bound to a moiety to result in a net reduction of positive charge in the compound. Capping groups can include cyclic anhydrides, carboxylic acids, activated esters, isothiocyanates, and isocyanates.

In some embodiments P is selected from: polylysine, PAMAM dendrimers, polyvinylacetic acid, polyacrylic acid, hyaluronic acid, glycosaminoglycans, and derivatized dextrans.

Multimeric CEST Chelating Ligands and Metal Chelate Agents

As can be seen from the genus of polymeric derivatives above, multimeric CEST chelating ligands can be prepared. Any of the CEST chelating ligands or chelates set forth above are amenable to the preparation of multimeric CEST metal chelates and contrast agents by covalently linking two or more of them to a multimeric scaffold (e.g., P above). A multimeric CEST contrast agent includes two or more CEST agents, which may be the same or different. The CEST effect is amplified as the CEST agents are linked together in a multimeric fashion, as there are more exchangeable hydrogens and/or the water exchange rate of multiple waters has been optimized. In some cases, the multimeric scaffold itself contains exchangeable hydrogens, which are also shifted, resulting in an additional CEST effect which can be realized by irradiating at the frequency of these exchanging hydrogens.

Suitable multimeric scaffolds are set forth in U.S. Pat. No. 6,652,835. For example, one multimeric CEST agent based on multimeric scaffolds set forth in '835 has the structure:

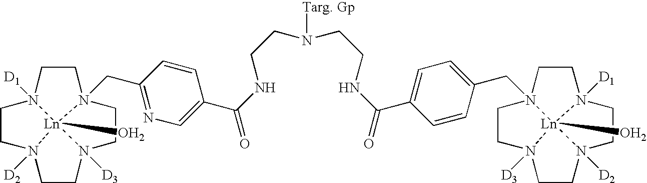

where “Targ. Gp” represents a TBM, and the Ds can be as described above.

Other suitable moieties for incorporating two or more CEST agents in the preparation of a multimeric CEST agent include the linker and linker subunit moieties set forth in U.S. Ser. No. 10/209,172, entitled “Peptide-Based Multimeric Targeted Contrast Agents,” filed on Jul. 30, 2002, and published as U.S. Publication US-2003-0216320-A1.

In addition, multimeric scaffold building blocks can include, but are not limited to, poly-lysine, polyornithine, poly-diaminobutyric acid, poly-arginine, or other multimeric natural and unnatural amino acids. (See, e.g., polymeric derivatives above). The multimeric scaffold backbone could also be a peptide containing several exchangeable hydrogens from amide N—H protons, and sidechains with exchangeable amine N—H, amide N—H, alcohol O—H, or amidine N—H protons. Alternatively, the scaffold could be constructed using a dendrimer, wherein the dendrimer contains exchangeable hydrogens. Oligo-saccharide scaffolds such as polydextran could also be derivatized with CEST agents and the exchangeable —OH groups of the sugars exploited for the CEST effect.

Coupling to a scaffold typically uses standard organic chemistry coupling procedures, as indicated previously. Coupling may introduce asymmetry in the molecule, but this should not modify the magnetic susceptibility tensor to result in largely different chemical shifts for otherwise equivalent exchangeable protons. This should permit simultaneous irradiation, particularly given the broadband irradiation pulses that can be programmed on an MRI system.

Multimeric CEST agents can include one or more target binding moieties (TBMs), as described in U.S. Pat. No. 6,652,835, U.S. Publication US-2003-0216320 A1, and as set forth more fully below. A TBM can be covalently linked (optionally through a linker) to one or more CEST chelates, to one or more positions on the scaffold, or some combination of the two. A TBM, such as a peptide TBM, can target the multimeric CEST agent to a target in vivo, such as a component of the heart (e.g., myocardium) or brain.

Synthesis

Chelating ligands can be synthesized by methods known in the art. See, e.g., U.S. Pat. Nos. 6,406,297 and 6,515,113; U.S. Ser. No. 60/466,238, entitled “Chelating Ligands,” filed Apr. 28, 2003, and U.S. Ser. No. 60/466,452, entitled “Agents and Methods for Myocardial Imaging,” filed Apr. 28, 2003, all of which are incorporated herein by reference.

Targeting Groups

Chelating ligands may be modified to incorporate one or more Target Binding Moieties (TBM), as indicated above. TBMs can include peptides, nucleic acids, or small organic molecules. TBMs allow chelating ligands and metal chelates to be bound to targets in vivo. Typically, a TBM has an affinity for a target. For example, the TBM can bind its target with a dissociation constant of less than 10 μM, or less than 5 μM, or less than 1 μM, or less than 100 nM. In some embodiments, the TBM has a specific binding affinity for a specific target relative to other physiologic targets. For example, the TBM may exhibit a smaller dissociation constant for collagen relative to its dissociation constant for fibrin.

TBMs can be synthesized and conjugated to the chelating ligands by methods well known in the art, including standard peptide and nucleic acid synthesis methods; see, e.g., WO 01/09188, WO 01/08712, and U.S. Pat. Nos. 6,406,297 and 6, 515,113. Typically, a TBM is covalently bound to the chelating ligand, and can be covalently bound to the chelating ligand through an optional Linker (L). As indicated in the structures above, a TBM may be anywhere on a chelating ligand. For example, the TBM may be bound, optionally via a L, to an ethylene group on the tetraazacyclododecane backbone, or to the ethylene C atoms of any acetate groups on the chelating ligand, or to any Ds on the backbone, as shown below:

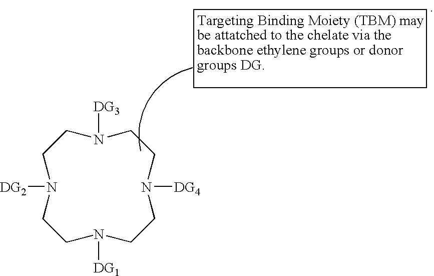

Typical targets include human serum albumin (HSA), fibrin, an extracellular component of myocardium (e.g., collagen, elastin, and decorin), or an extracellular component of a lesion (e.g., hyaluronic acid, heparin, chondroitin sulfate, dermatan sulfate, heparan sulfate, keratan sulfate, versican, and biglycan).

Linkers

In some embodiments, a TBM can be covalently bound to a chelating ligand through a linker (L). The L can include, for example, a linear, branched or cyclic peptide sequence. In one embodiment, a L can include the linear dipeptide sequence G-G (glycine-glycine). In embodiments where the TBM includes a peptide, the L can cap the N-terminus of the TBM peptide, the C-terminus, or both N- and C- termini, as an amide moiety. Other exemplary capping moieties include sulfonamides, ureas, thioureas and carbamates. Ls can also include linear, branched, or cyclic alkanes, alkenes, or alkynes, and phosphodiester moieties. The L may be substituted with one or more functional groups, including ketone, ester, amide, ether, carbonate, sulfonamide, or carbamate functionalities. Specific Ls contemplated include NH—CO—NH—; —CO—(CH₂)ⁿ—NH—, where n=1 to 10; dpr; dab; —NH—Ph-; —NH—(CH₂)ⁿ—, where n=1 to 10; —CO—NH—; —(CH₂)ⁿ—NH—, where n=1 to 10; —CO—(CH₂)ⁿ—NH—, where n=1 to 10; 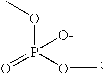
and —CS—NH—. Additional examples of Ls and synthetic methodologies for incorporating them into chelating ligands, particularly chelating ligands comprising peptides, are set forth in WO 01/09188, WO 01/08712, and U.S. patent application Ser. No. 10/209,183, entitled “Peptide-Based Multimeric Targeted Contrast Agents,” filed Jul. 30, 2002.

Properties of Chelating Ligands and Metal Chelates

The chelating ligands described above are capable of binding one or more metal ions to result in a metal chelate, e.g., a metal chelate useful as a CEST agent. Metal chelates can be prepared by methods well known in the art; see WO 96/23526, U.S. Pat. Nos. 6,406,297 and 6,515,113. Metal chelates can include lanthanide metal ions such as Dy(III), Ho(III), Er(III), Pr(III), Eu(III), Nd(III),Tb(III), Tm(III), Ce(III), and Yb(III). Typically, because of the chemical nature and number of Ds on the chelating ligands, the metal ion is tightly bound by the chelating ligand, and physiologically compatible metal chelates can be made. The formation constant, K^{f}, of a chelating ligand for a metal ion is an indicator of binding affinity, and is typically discussed with reference to a log K^{f} scale. Physiologically compatible metal chelates can have a log K^{f} ranging from 15 to about 25 M⁻¹. Methods for measuring Kf are well known in the art; see, e.g., Martell, a. E., Motekaitis, R. J., Determination and Use of Stability Constants, 2d Ed., VCH Publishers, New York (1992).

Luminescence lifetime measurements can be used to evaluate the number of water molecules bound to a metal chelate. Methods for measuring luminescence lifetimes are known in the art, and typically include monitoring emissive transitions of the chelate at particular wavelengths for lifetime determination, following by fitting of luminescence decay data. Luminescence lifetime measurements are also useful for evaluating the suitability of the metal chelates as luminescent probes.

Metal chelates of the invention can also be screened to determine efficacy as chemical exchange saturation transfer (CEST) contrast agents. Water exchange rates (water residency times) can be used as one indicator of useful CEST agents. Metal chelates can thus be evaluated for the mean residence time of water molecule(s) in the first (or higher) coordination sphere(s). The mean residence time of water molecules is the inverse of the water exchange rate and is dependent on temperature. ₁₇O NMR can be used to evaluate the mean residence time of water molecules by methods known to those of ordinary skill in the art. Water residency times of 1000 ns and longer of a Gd(III) metal chelate can indicate that the chelating ligand is useful as a CEST contrast agent with other lanthanide(III) ions, including Yb, Ce, Tm, Er, Ho, Dy, Th, Eu, Pr, and Nd. See, for example, U.S. Ser. No. 60/466,238, entitled “Chelating Ligands,” filed Apr. 28, 2003, incorporated herein by reference.

Use of Chelating Ligands and Metal Chelates

Chelating ligands can be used to prepare metal chelates, as described above, for diagnostic purposes. For example, metal chelates can be useful as CEST contrast agents in MR imaging. Contrast agents incorporating a TBM can bind a target and therefore can be particularly useful in targeted MR applications, e.g., to image reduced blood flow and volume as a result of clots. Preferably at least 10% (e.g., at least 50%, 80%, 90%, 92%, 94%, or 96%) of the contrast agent can be bound to the desired target at physiologically relevant concentrations of contrast agent and target. The extent of binding of a contrast agent to a target can be assessed by a variety of equilibrium binding methods, e.g., ultrafiltration methods; equilibrium dialysis; affinity chromatography; or competitive binding inhibition or displacement of probe compounds.

Metal chelates of lanthanides can also be useful as luminescent probes. Luminescent metal chelate probes can be useful in a variety of assays, e.g., to detect, separate, and/or quantify chemical and biological analytes in research and diagnostic applications, including high-throughput, real-time, and multiplex applications. For example, probes incorporating a TBM can bind to a target analyte of interest, and can have long luminescent lifetimes (e.g., greater than 0.1 μs, or 100 μs, or 1 ms), thereby improving sensitivity and applicability of various assay formats. See, generally, U.S. Pat. Nos. 6,406,297 and 6,515,113, for a description of assays suitable for inclusion of luminescent metal chelate probes. Luminescent metal chelate probes are particularly useful in immunoassays and real-time PCR detection assays.

Methods

Perfusion and Blood Volume Changes in Heart and Brain

The invention also provides methods for measuring perfusion and blood volume changes in the heart and brain. For evaluating perfusion in the heart, the methods described herein rely on the change in blood volume in the heart upon going from a resting state to a stress state (e.g., a hyperemic state), such as through exercise or a pharmacologic stressor. In the heart, blood volume increases by a factor of about two upon going from a resting state to a state of hyperemia. While narrowed arteries can deliver sufficient blood volume during rest, under the increased stress, not enough blood volume can be delivered and the tissue fed by the narrowed arteries becomes ischemic. By comparing blood volume at stress and rest, it is possible to identify ischemic areas.

Any chemical exchange saturation transfer (CEST) contrast agent, such as the ones described above or in the literature, can be used to measure blood volume. A CEST image can include the acquisition of two images: one image is acquired with a saturation pulse applied at the frequency of the exchangeable hydrogen (+ω¹) and then the same image is acquired with a saturation pulse applied at a different frequency. In certain cases, the different frequency is −ω¹, but in theory, any other frequency than +ω¹ can be used, including frequencies of 2ω, 3ω, 0.5ω, 1.5ω, −2ω, −3ω, −0.5ω, and −1.5ω. The difference image of the two image gives a measure of the effect due to the CEST agent. As used herein, such a difference image is termed a “CEST image.” The image acquired with a saturation pulse at the different frequence (e.g., −ω¹) is used as a baseline and includes any magnetization transfer effects arising from tissue. Such an experiment can be done with an interleaved pulse sequence (e.g., where the +ω¹ and −ω¹ are alternated) to minimize any motion artifacts from the subtraction image.

The invention provides a method to determine a change in blood volume in one or more areas of a heart (e.g., of a mammal, such as a human) between a rest state and a stress state (e.g., hyperemia induced through exercise or through the use of a pharmacological stressor). Hyperemia, or peak hyperemia, refers to the point approaching maximum increased blood supply to an organ or blood vessel for physiologic reasons. Exercise-induced hyperemia can be achieved through what is commonly known as a “stress test” and has several clinically relevant endpoints, including excessive fatigue, dyspnea, moderate to severe angina, hypotension, diagnostic ST depression, or significant arrhythmia. Pharmacologic stressors include vasodilators, such as dobutamine or Dipyridamole (Persantine™).

The method includes administering a CEST-contrast agent to the mammal, such as by i.v. injection. The CEST agent may be allowed to reach a steady state concentration in the blood. A first CEST image of the heart (e.g., a rest CEST image) can then be acquired, as described above. To acquire this image, the mammal is positioned inside an MRI machine. The mammal can then be put in a stress state. For example, a pharmacological stress agent (such as Dipyridamole or dobutamine) can be administered to increase blood flow (and concomitantly, blood volume). A second CEST image (e.g., a stress CEST image) is then acquired during the period of stress. The two CEST images are then compared and/or combined (e.g., by subtraction or division). By combining the two CEST images, an image reflective of blood volume change in one or more areas of the heart is obtained. Regions (areas) with large differences between the two CEST images indicate normal tissue, while regions (areas) with small differences between the two CEST images represent ischemic tissue (or tissues exhibiting small blood volume changes). As the concentration of the CEST agent does not change very much over the period of time required to obtain the images, differences between the two CEST images are due to blood volume changes in the area of the heart.

CEST agents can also be used to image cerebral blood volume changes in areas of the brain, including increases and decreases of blood volume in the brain. For example, a similar method as outlined above can be used to determine regions of ischemia in areas of the brain (e.g., stroke); to diagnose or evaluate various brain disorders, such as Alzheimer's disease, schizophrenia, or bipolar disorder; or to evaluate brain function (e.g., fMRI using CEST). In brain imaging, blood flow and blood volume can be increased in a similar manner as in the heart, e.g., the induction of a “stress state” such as by exercise or administration of a pharmacologic stressor such as an antipsychotic drug. In addition, increased flow to regions of the brain can be induced by a visual stimulus, an auditory stimulus, an olfactory stimulus, a tactile stimulus, a gustatory stimulus, or any of the stimuli or methods conventionally used in fMRI or brain PET studies. As used herein, these stimuli or methods are also referred to as inducing a “stress state.” Other stressors result in decreased blood flow and blood volume to the brain and their effect can also be analyzed using the methods provided herein. Measuring blood volume changes in the brain with a CEST contrast agent may provide greater sensitivity and hence better diagnostic accuracy than prior fMRI or PET studies.

In the method, a CEST-contrast agent is administered to a mammal, such as by i.v. injection. The CEST agent may be allowed to reach a steady state concentration in the blood. A first CEST image of the brain (e.g., a rest CEST image) can then be acquired, as described above. To acquire this image, the mammal is positioned inside an MRI machine. The mammal can then be put in a stress state. A stress state in the brain to result in increased blood volume can be induced by hyperthermia, exercise, or administration of a pharmacological stress agent. Suitable pharmacologic stress agents to increase blood volume include antipsychotic drugs such as phenothiazines, e.g., chlorpromazine, thioridazine, and trifluoperazine; and various other medications including haloperidol, thiophixene, lithium; acetazolamide; and ketamine. In other cases, the mammal can be exposed to a stimulus (e.g., an olfactory stimulus) to increase blood flow, as described previously. A stress state can also result in reduced blood volume in the brain, such as the result of hypothermia or the administration of a pharmacologic stress agent such as barbiturates, caffeine, propofol, etiomidate, and lidocaine. A second CEST image (e.g., a stress CEST image) is then acquired during the period of stress. The two CEST images are then compared and/or combined. By combining the two CEST images, an image reflective of blood volume change is obtained. Regions with large differences between the two CEST images indicate normal tissue, while regions with small differences between the two CEST images represent ischemic tissue or tissues exhibiting small blood volume change. As the concentration of the CEST agent does not change very much over the period of time required to obtain the images, differences between the two CEST images are due to blood volume changes or specifically activated brain tissue.

Any of the methods described above can be altered in the sequence of stress and rest. Thus, for example, a CEST image at stress can be followed by a CEST image at rest.

Imaging of Sparse Epitopes, Including Microthrombi CEST agents can be targeted to specific disease states by conjugating a specific protein binding moiety to a CEST agent. For example, CEST agents can bind to thrombin by linking the CEST agent to a fibrin binding peptide. The on/off nature of the CEST effect allows for signal averaging, which may be particularly usefuil when imaging sparse epitopes, such as microthrombi.

In one embodiment, the invention provides methods and CEST agents for imaging microthrombi in the brain. Typically, such CEST agents are targeted to fibrin and can be monomeric or multimeric (e.g., include two or more CEST metal chelates).

Because a CEST agent only gives contrast when the correct pulse sequence is employed, one can use it as an on/off agent. By using a pulse-sequence where the saturating pulse on the exchangeable hydrogen (ω)) is interleaved with one where there is a pulse at a frequency different (e.g., opposite that of the exchangeable hydrogen (ω))), one generates two images. The difference of these two images is an image with contrast given by the CEST agent (“CEST image”). If this process is repeated and the difference images averaged, then the signal from the CEST agent will add and the noise will cancel out. In such a way it is possible to detect small changes in signal, such as when a fibrin targeted CEST agent is bound to microthrombi in the brain.

In the method, a CEST-contrast agent is administered to a mammal, such as by i.v. injection. The CEST agent may be allowed to reach a steady state concentration in the thrombus and/or blood. A CEST image of the brain can then be acquired, as described above. To acquire this image, the mammal can be positioned inside an MRI machine. In certain cases, a thrombolytic can be administered, e.g., after the acquisition of the CEST image. A CEST image taken before administration of a thrombolytic can be compared with a CEST image taken after administration of a thrombolytic in order to evaluate efficacy of the thrombolytic.

Use of CEST Contrast Agents of the Invention

Some CEST contrast agents may provide multiple, distinct resonance peaks for magnetization transfer (e.g., both —OH and —NH groups). In order to maximize the MT effects (and thus the efficiency of the CEST agent for providing MR contrast), sequence modifications to saturate the multiple peaks may be beneficial. These can be achieved by using (a) multiple pulses at different center frequencies, (b) single pulses with complex amplitude or phase modulation in order to create the frequency content for two or more MT resonances, or (c) continuous excitation at combined frequencies. Frequencies above and below the primary water resonances can also be used to uniquely identify the CEST effect from other off-resonance phenomena using these techniques as well. Alternatively, MT effects from the individual chemical groups can be combined (e.g., resulting images combined by adding, multiplication, or other techniques available to those skilled in the art) by combining multiple acquisitions each optimized to the individual frequencies. For example, for a two group combination, which has two chemical shifts, δ1 and δ2, the following notation can be adopted: Iω=intensity of image with MT irradiation at frequency w offset from water; I(ω1,ω2)=intensity of image with combined MT irradiation at both frequencies. One can make a CEST image by comparing I(δ1,δ2) to I(−δ1 ,−δ2) or combining I(−δ1)/I(−δ1) and I(−δ2)/I(−62). Other combinations of off-resonance excitations that isolate or intensify the MT effects for the multiple resonances can be deduced by those skilled in the art.

Pharmaceutical Compositions

Contrast agents of the invention can be formulated as a pharmaceutical composition in accordance with routine procedures. As used herein, the contrast agents of the invention can include pharmaceutically acceptable derivatives thereof. “Pharmaceutically acceptable” means that the agent can be administered to an animal without unacceptable adverse effects. A “pharmaceutically acceptable derivative” means any pharmaceutically acceptable salt, ester, salt of an ester, or other derivative of a contrast agent or compositions of this invention that, upon administration to a recipient, is capable of providing (directly or indirectly) a contrast agent of this invention or an active metabolite or residue thereof. Other derivatives are those that increase the bioavailability when administered to a mammal (e.g., by allowing an orally administered compound to be more readily absorbed into the blood) or which enhance delivery of the parent compound to a biological compartment (e.g., the brain or lymphatic system) thereby increasing the exposure relative to the parent species. Pharmaceutically acceptable salts of the contrast agents of this invention include counter ions derived from pharmaceutically acceptable inorganic and organic acids and bases known in the art.

Pharmaceutical compositions of the invention can be administered by any route, including both oral and parenteral administration. Parenteral administration includes, but is not limited to, subcutaneous, intravenous, intraarterial, interstitial, intrathecal, and intracavity administration. When administration is intravenous, pharmaceutical compositions may be given as a bolus, as two or more doses separated in time, or as a constant or non-linear flow infusion. Thus, contrast agents of the invention can be formulated for any route of administration.

Typically, compositions for intravenous administration are solutions in sterile isotonic aqueous buffer. Where necessary, the composition may also include a solubilizing agent, a stabilizing agent, and a local anesthetic such as lidocaine to ease pain at the site of the injection. Generally, the ingredients will be supplied either separately, e.g. in a kit, or mixed together in a unit dosage form, for example, as a dry lyophilized powder or water free concentrate. The composition may be stored in a hermetically sealed container such as an ampule or sachette indicating the quantity of active agent in activity units. Where the composition is administered by infusion, it can be dispensed with an infusion bottle containing sterile pharmaceutical grade “water for injection,” saline, or other suitable intravenous fluids. Where the composition is to be administered by injection, an ampule of sterile water for injection or saline may be provided so that the ingredients may be mixed prior to administration. Pharmaceutical compositions of this invention comprise the contrast agents of the present invention and pharmaceutically acceptable salts thereof, with any pharmaceutically acceptable ingredient, excipient, carrier, adjuvant or vehicle.

A contrast agent is preferably administered to the patient in the form of an injectable composition. The method of administering a contrast agent is preferably parenterally, meaning intravenously, intra-arterially, intrathecally, interstitially or intracavitarilly. Pharmaceutical compositions of this invention can be administered to mammals including humans in a manner similar to other diagnostic or therapeutic agents. The dosage to be administered, and the mode of administration will depend on a variety of factors including age, weight, sex, condition of the patient and genetic factors, and will ultimately be decided by medical personnel subsequent to experimental determinations of varying dosage followed by imaging as described herein. In general, dosage required for diagnostic sensitivity or therapeutic efficacy will range from about 0.001 to 50,000 μg/kg, preferably between 0.01 to 25.0 μg/kg of host body mass. The optimal dose will be determined empirically following the disclosure herein.

### EXAMPLES

### Example 1

### Preparation of Europium Complex of 10-[(4-(Methoxycarbonyl)-2-pyridinyl)methyl[-1,4,7-tris(4-carboxy-2-oxo-3-azabutyl)-1,4,7,10-tetraazacyclododecane

1a) Methyl 2-bromomethylnicotinate

1.0 g (5.98 mmol) Methyl 2-hydroxymethylnicotinate (J. Med. Chem. 1976, 19, 483) was dissolved in 25 mL of anhydrous tetrahydrofuran (THF) under argon. 0.85 mL (8.97 mmol) of phosphorotribromide were added under stirring at room temperature and the mixture was heated to 65° C. within 15 min. The brownish solution was then cooled to 5-10° C. and adjusted to pH 7 with saturated aqueous sodium hydrogencarbonate solution. It was extracted three times with ethyl acetate, washed with brine and dried over sodium sulfate. The product was purified by flash chromatography (hexane/ethyl acetate gradient from 3:1 to 1:1) to give 947 mg (69%) of a deep purple oil.

Elemental analysis:

| | | | | |
|---|---|---|---|---|
| calc.: | C 41.77 | H 4.83 | Br 34.73 | N 6.09 |
| found: | C 41.47 | H 4.92 | Br 34.22 | N 5.84 |

1b) 1,4,7-Tris[4-(t-butyloxycarbonyl)-2-oxo-3-azabutyl]-1,4,7,10-tetraazacyclododecane

6.57 g (26.06 mmol) of bromoacetyl-glycine t-butyl ester (H. Schmitt-Willich et al, Ger. Offen. (1998), DE 19652386 A1, example 11a) was dissolved in 80 mL acetonitrile. After addition of 1.5 g (8.68 mmol) cyclen, the mixture was stirred for 5 days. The solvent was removed in vacuo, the residue was taken up in dichloromethane and extracted three times with water, washed with brine and dried over magnesium sulfate. The product was purified by flash chromatography (dichloro methane/methanol gradient from 15:1 to 3:1 with 1% of triethylamine) to give 2.25 g (37.8%) of a white powder.

Elemental analysis:

| | | | |
|---|---|---|---|
| calc.: | C 56.04 | H 8.67 | N 14.30 |
| found: | C 55.87 | H 8.80 | N 14.49 |

1c) 10-[(4-(Methoxycarbonyl)-2-pyridinyl)methyl]-1,4,7-tris[4-(t-butyloxacarbonyl)-2-oxo-3-azabutyl]-1,4,7,10-tetraazacyclododecane

3.2 g (4.67 mmol) of 1,4,7-Tris[4-(t-butyloxycarbonyl)-2-oxo-3-azabutyl]-1,4,7,10-tetraazacyclododecane were dissolved in 120 mL anhydrous dichloromethane under argon. After addition of 1.15 g (5 mmol) of methyl 2-bromomethylnicotinate and 1.4 mL of triethylamine, the mixture was stirred overnight at room temperature. The brownish solution was extracted three times with saturated aqueous sodium hydrogen-carbonate solution, washed with brine and dried over sodium sulfate. The product (3.59 g) was used in the next reaction without further characterization.

1d) Europium Complex of 10-[(4-(Methoxycarbonyl)-2-pyridinyl)methyl]-1,4,7-tris(4-carboxy-2-oxo-3-azabutyl)-1,4,7,10-tetraazacyclododecane

2.1 g (2.5 mmol) of 10-[(4-(methoxycarbonyl)-2-pyridinyl)methyl]-1,4,7-tris[4-(t-butyloxycarbonyl)-2-oxo-3-azabutyl]-1,4,7,10-tetraazacyclododecane were treated with 10 mL of 1.2 N HCl/acetic acid. After stirring for 2 hours at room temperature, 100 mL of ether were added to the solution and the precipitate was filtered off and dried.

The obtained ligand was dissolved in water and adjusted to pH 5 by addition of 0.1 N NaOH. After addition of 646 mg (2.5 mmol) europium chloride, the mixture was stirred for 6 h at 50° C. The solution was freeze-dried and the lyophilisate was chromatographed on a RP-18 column using acetonitrile/water as eluent. The fractions that contained the product were combined and freeze-dried. Yield: 820 mg (37%), water content (Karl Fischer determination): 8.0%

Elemental analysis (referring to the water-free substance):

| | | | | |
|---|---|---|---|---|
| calc.: | C 41.23 | H 4.82 | N 13.74 | Eu 18.63 |
| found: | C 41.08 | H 4.90 | N 13.55 | Eu 18.22 |

Structure:

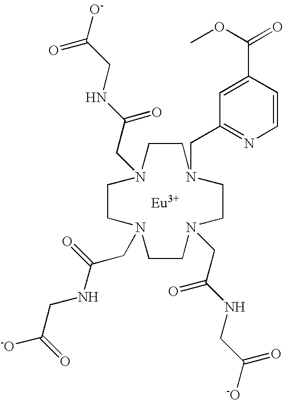

### Example 2

### Synthesis of the Dy(III) chelate of DTPA bis-N,N-bis(N-(tris(hydroxymethyl)methyl)carbamoylethyl)-amide

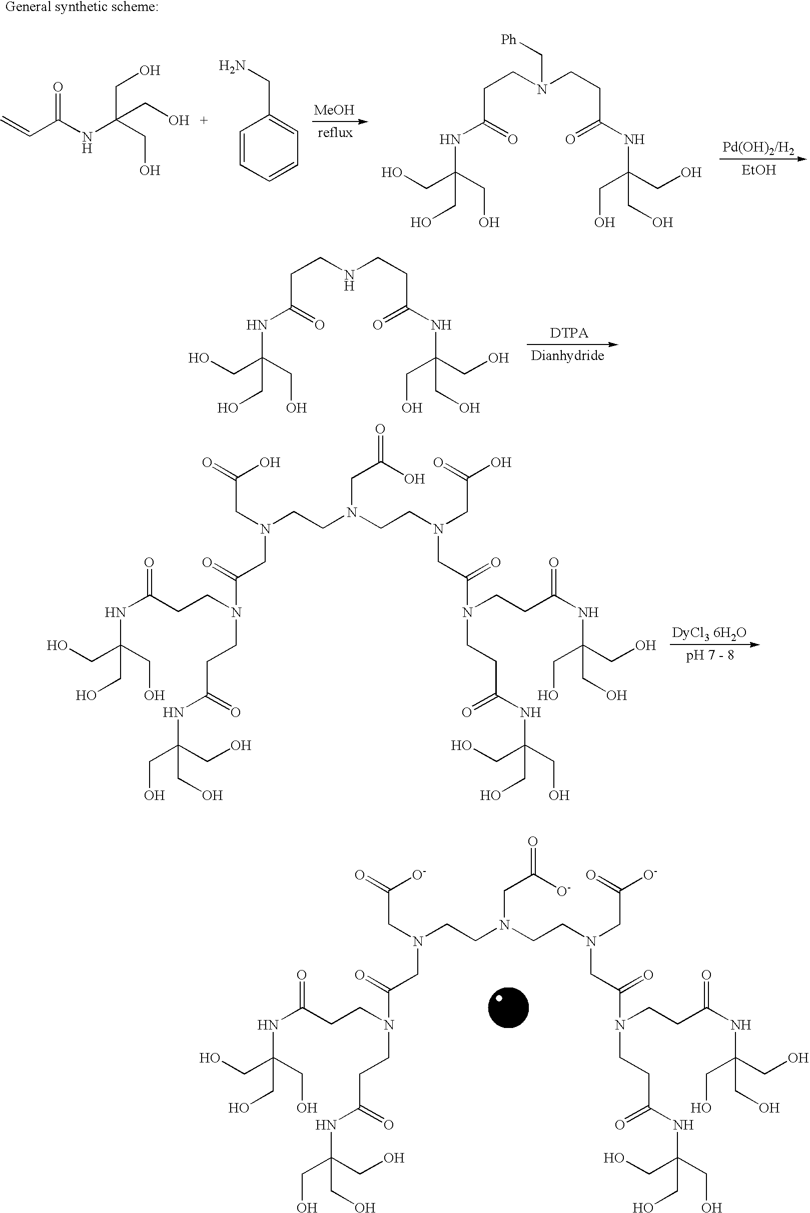

2a) Synthesis of N-benzyl-N,N-bis(N-(tris(hydroxymethyl)methyl)carbamoylethyl)amine

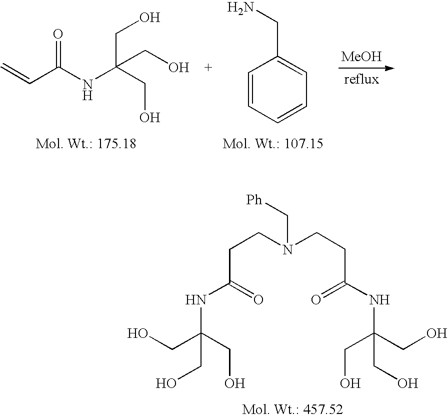

A solution of N-(tris(hydroxymethyl)methyl)acrylamide (7.7 g, 40.8 mmol, 93%) and benzylamine (1.07 g, 10 mmol) in 40 mL MeOH was heated at refluxing for 24 hr. After cooling to 25° C. the solvent was removed in vacuo, and the product was purified by silica gel flash column chromatography (eluent MeOH/CH₂Cl₂ (10/90-15/85-20/80); step gradient). Fractions containing pure bisamide were combined (R^{f}=0.2 MeOH/CH₂Cl₂: 10/90) to give 1.82 g of colorless viscous oil (40%). MS [M+H⁺]=458.5.

2b) Synthesis of N,N-bis(N-(tris(hydroxymethyl)methyl)carbamoylethyl)amine

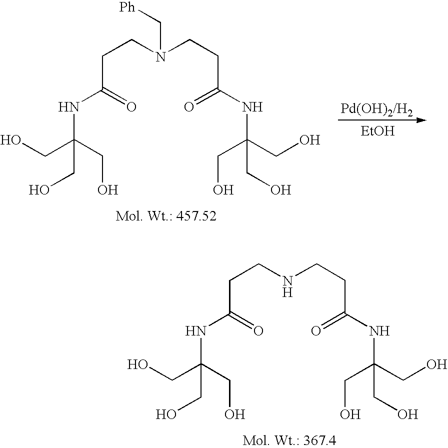

A mixture of the bisamide (0.93 g, 2.0 mmol) and palladium hydroxide (10% on charcoal) (0.4 g) in 30 mL EtOH was hydrogenated under 48 psi of H₂ with a Parr hydrogenation instrument for 4 hr. Palladium hydroxide/charcoal was filtered off through a pad of celite in a sintered glass funnel. The celite was rinsed with EtOH (10 mL×2). The filtrate, combined with the rinse, was concentrated in vacuo to yield 0.75 g (100%) of the secondary amine. MS [M+H⁺]=368.3.

2c) Synthesis of DTPA bis-N,N-bis(N-(tris(hydroxymethyl)methyl)carbamoylethyl)-amide

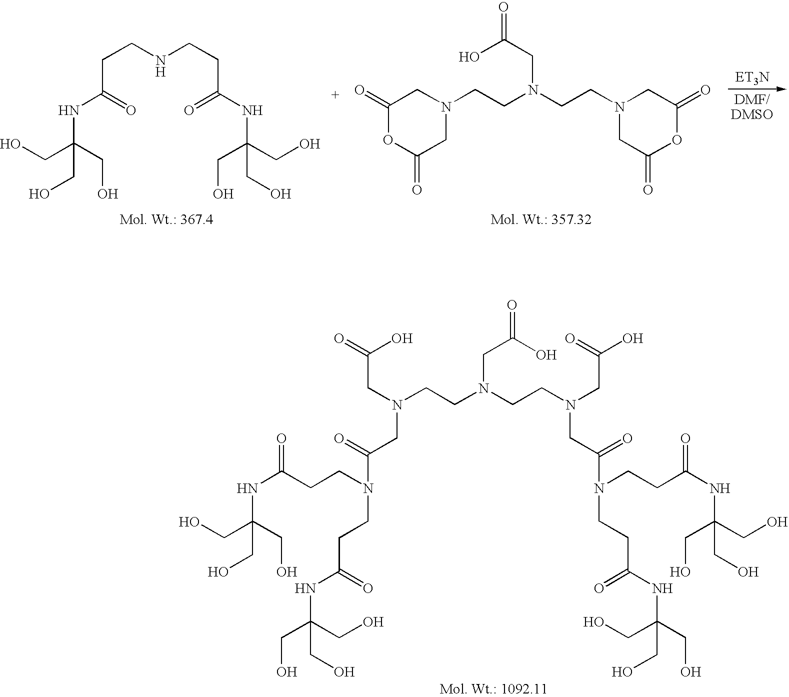

A solution of the secondary amine (0.75 g, 2.0 mmol) in 2 mL DMF was added dropwise over 10 min to a stirred solution of DTPA dianhydride (0.29 g, 0.8 mmol) and Et₃N (0.30 g, 2.9 mmol) in 5 mL DMSO at 22° C. The reaction was stirred for an additional 24 hr and then poured into 60 mL dioxane. The precipitate was collected by filtration and separated on a Dowex-H⁺ (6×1 in) column by applying a gradient from 0-100 mM formic acid solution. The fractions containing the desired product were collected and freeze-dried. MS [M+H₊]=1093.3.

2d) Synthesis of the Dy(III) chelate of DTPA bis-N,N-bis(N-(tris(hydroxymethyl)methyl)carbamoylethyl)-amide

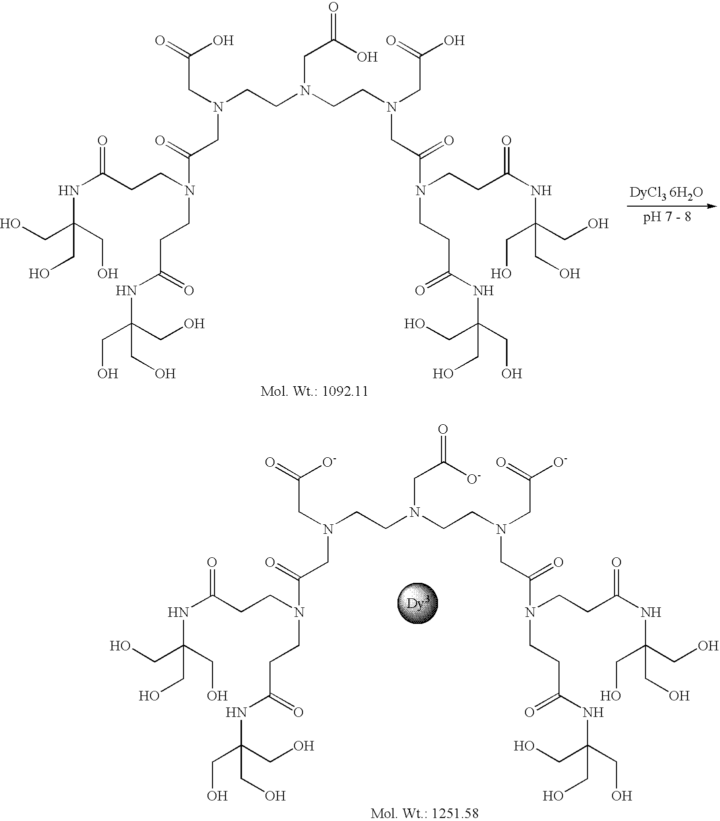

DyCl₃.6H₂O (105 mg, 0.27 mmol) was added to a solution of DTPA-bisamide (140 mg, 0.13 mmol) in 5 mL H²O. Sodium hydroxide (1M solution in water) was added to the solution to bring the pH to 7-8. The reaction was stirred for 15 hr and the precipitate formed during the reaction was removed by filtration. The chelate was purified by preparative HPLC method, which used a Rainin Dynamax SD-1 HPLC system, a Rainin Dynamax VU-1 detector (λ=220 nm), and an Akzo Nobel Kromasil C₁₈ preparative column (20 mm×250 mm, 10 μm particle size, 100 Å pore size). The column was eluted with isocratic EtOH/H₂O (2/98) with a flow rate of 18 mL/min for 12 min. Fractions containing pure Dy-chelate were combined and freeze-dried to yield 64 mg (40%) of a white powder. MS [M+H₊]=1251.4, 1252.2, 1253.4, 1254.4.

### Example 3

### Preparation of 1,4,7,10-Tetraazacyclododecane-1,7-bis(carboxymethyl)-4,10-bis[2-(R)-pentanedioic acid, 5-tris(hydroxymethyl)aminomethane] (5)

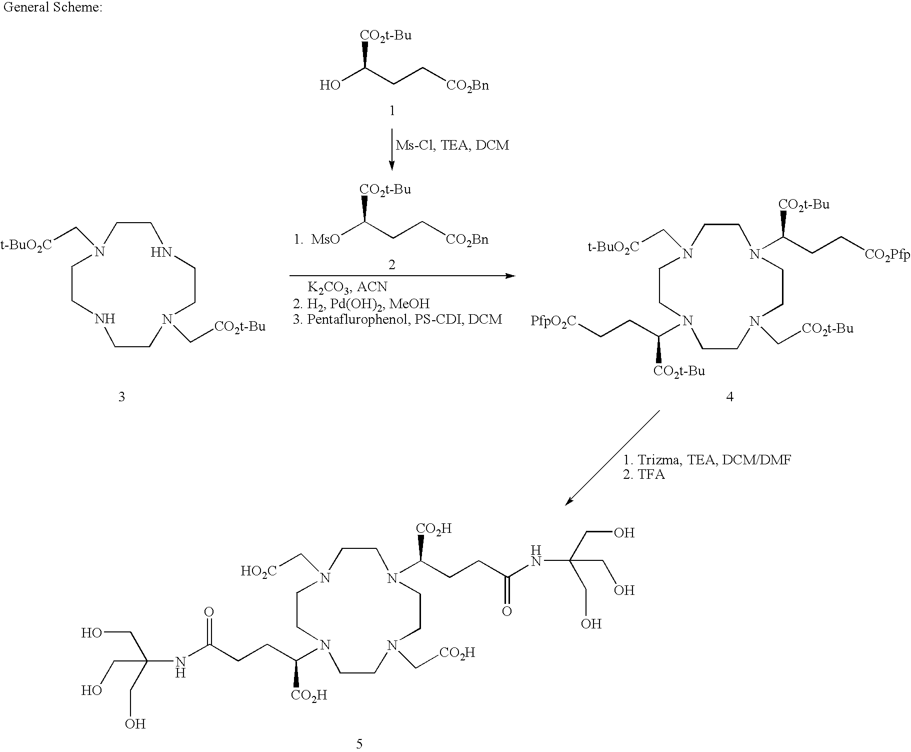

3a) 2-(S)-2-Mesyloxy pentanedioic acid, 1-t-butyl-5-benzyl ester (2)

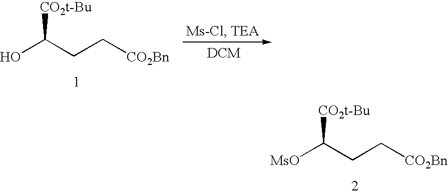

Methanesulfonyl chloride (6.5 mL, 80 mmol) was added to a stirred mixture of 2-(S)-2-hydroxy pentanedioic acid, 1-t-butyl-5-benzyl ester (1) (22.6 g, 77 mmol) and NEt₃ (11.5 mL, 82.5 mmol) in CH₂Cl₂ (200 mL) cooled to 0-5° C. in an ice bath. After the addition was complete, the mixture was warmed to room temperature. Water (300 mL) was added; the organic phase was separated and dried (Na₂SO₄), decanted and concentrated in vacuo to give 29.0 g (100%) of 2. MS [M+Na]=395.

3b) 1,4,7,10-Tetraazacyclododecane-1,7-bis(t-butyloxycarbonyl-methyl)-4,10-bis[2-(R)-pentanedioic acid, 1-t-butyl-5-benzyl ester] (6)

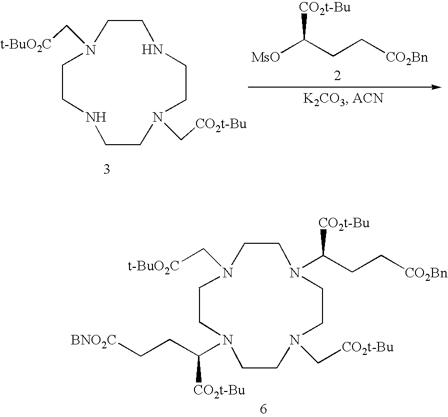

K₂CO₃ (0.8 g, 5.8 mmol) was added to a solution of DO2A t-butyl ester (3) (0.4 g, 1 mmol) and mesylate 2 (0.9 g, 2.4 mmol) in acetonitrile (20 mL). The mixture was stirred at room temperature. NEt₃ (0.5 ml) was then added and the solution was heated at 80° C. The reaction was monitored by LC-MS. Additional NEt₃ was added and the reaction was extended. Crude 6 was purified on a flash column eluted by CH₂Cl₂:MeOH:TEA (99:1:0.2 to 90:10:0.2). Two fractions were collected, pooled and evaporated to leave 0.47 g of pure 6. MS [M+1]=953.

3c) 1,4,7,10-Tetraazacyclododecane-1,7-bis(t-butyloxycarbonyl-methyl)-4,10-bis[2-(R)-pentanedioic acid, 1-t-butyl ester] (7)

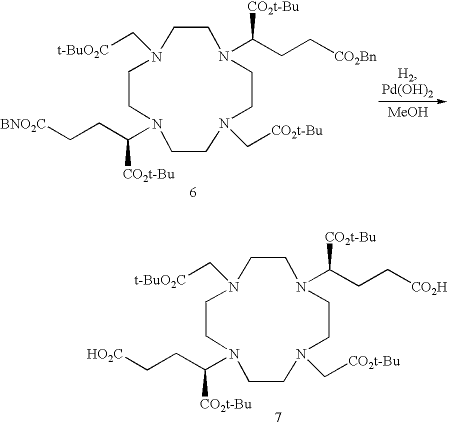

Benzyl ester (6) was dissolved in MeOH (20 mL) in a Parr bottle. Pd(OH)₂ was added. The mixture was hydrogenated at 50 psi. It was filtered through celite and concentrated in vacuo. Methanol was removed by co-evaporation with acetonitrile (2×20 mL). Diacid 7 was isolated in quantitative yield. MS [M+1]=773.

3d) 1,4,7,10-Tetraazacyclododecane-1,7-bis(t-butyloxycarbonyl-methyl)-4,10-bis[2-(R)-pentanedioic acid, 1-t-butyl-5-pentafluorophenyl ester] (4)

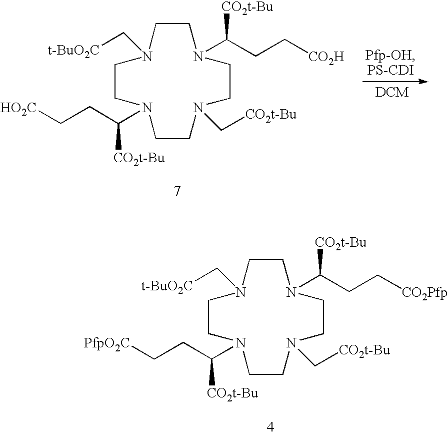

Acid 7 was dissolved in CH₂Cl₂. Pentafluorophenol (1.5 equiv.) and polystyrene-supported carbodiimide (Argonaut Technologies, 2 equiv.) were added. The mixture was shaken for 2 hours. The solution was filtered to remove the solid-supported reagent, which was washed with excess CH₂Cl₂. The solution was concentrated in vacuo to give compound 4 in quantitative yield. MS [M+1]=1105.

3e) 1,4,7,10-Tetraazacyclododecane-1,7-bis(t-butyloxycarbonyl-methyl)-4,10-bis[2-(R)-pentanedioic acid, 1-t-butyl ester, 5-tris(hydroxymethyl)aminomethane] (8)

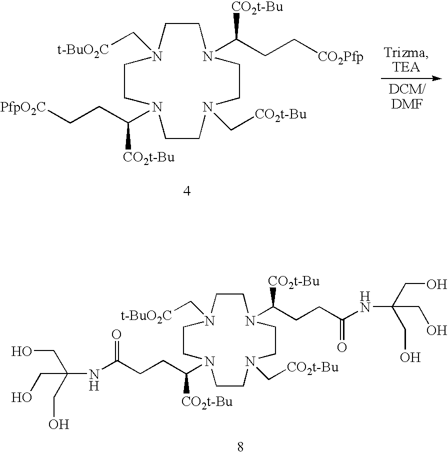

Tris-hydroxymethyl-aminomethane (Trizma, 4 equiv) is added to a solution of 4 in a mixture of CH₂Cl₂ and DMF. The mixture is stirred at room temperature. NEt₃ is then added. When the reaction is complete, the mixture is filtered. The solid is washed with more solvent and the solution is concentrated in vacuo. Crude 8 is purified by flash chromatography. MS [M+1]=980

3f) 1,4,7,10-Tetraazacyclododecane-1,7-bis(carboxymethyl)-4,10-bis[2-(R)-pentanedioic acid, 5-tris(hydroxymethyl)aminomethane] (5)

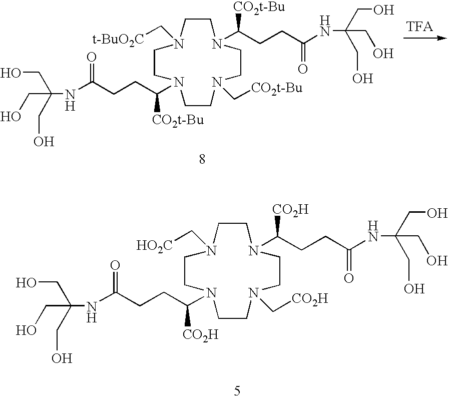

t-Butyl ester 8 is placed in a round-bottom flask. The flask is cooled in an ice-water bath and a cocktail of TFA, methanesulfonic acid and phenol (95:2.5:2.5 v/v/v) is added. The mixture is stirred and allowed to warm up to room temperature. After two hours, it is carefully poured into ether. The precipitate that forms is separated by centrifugation and washed three times with fresh ether. It is dried under vacuum. MS [M+1]=756.

### Example 4

### Polylysine conjugate with the europium complex of 1,4,7,10-tetra-[N-(carboxymethyl)-carbamoyl-methyl]1,4,7,10-tetraaza-cyclododecane

### Example 4a

### Preparation of polylysine conjugate with the europium complex of 1,4,7,10-tetra-[N-(carboxymethyl)-carbamoyl-methyl]1,4,7,10-tetraaza-cyclododecane

The europium complex of 1,4,7,10-tetra-[N-(carboxymethyl)-carbamoyl-methyl]1,4,7,10-tetraaza-cyclododecane (S. Aime et al, Magn Reson Med 2002, 47, 639) is coupled with poly-lysine in DMF in a 1:1 molar ratio of the complex/lysyl residue of the poly-lysine using TBTU as activating agent and an excess of DIEA as base. After 24 h an excess of diglycolic acid anhydride is added as a solid powder and the mixture is stirred for another 24 h. Thereafter, the organic solvent is evaporated, the residue is taken up in water and the resulting solution is purified by ultrafiltration (membrane cut off 3,000 Da). The retentate is then lyophilized to give the title compound as a colourless fluffy powder.

### Example 4b

### CEST Spectrum of Eu-Polylysine Derivative (Example 4a)

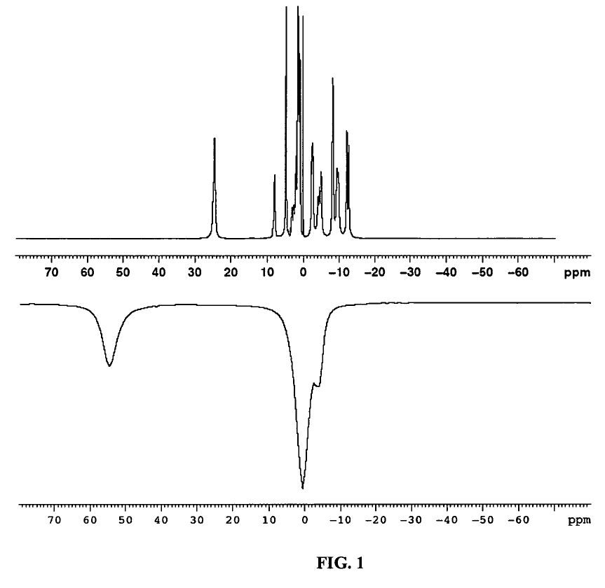

FIG. 1 shows the ₁H NMR (top) and CEST (bottom) spectra in H₂O/D₂O of the Eu-polylysine derivative described in Example 4a. Spectra were recorded at room temperature, and 400 MHz (Bruker Avance 400 spectrometer). The CEST spectrum was recorded using a protocol similar to that described in Ward et al, J. Magn. Reson. 2000 143, 79. The CEST spectrum shows the intensity of the bulk water peak as a function of saturation frequency. Irradiation of the bound water resonance around 55 ppm results in a large decrease of the intensity of the bulk water peak. Similar observation is made when directly saturating the bulk water at 0 ppm and the exchangeable amide protons at −5 ppm.

### Example 4c

### Mouse Imaging with Eu-polylysine Derivative (Example 4a)

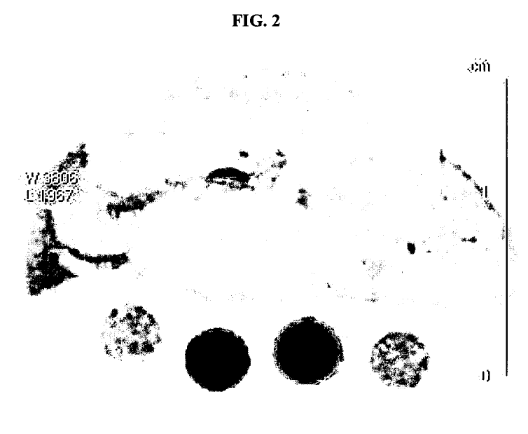

FIG. 2 shows a digital difference axial image between control image with saturation centered at −52 ppm (−10400 Hz) from the water resonance and CEST image with saturation centered at +52 ppm (10400 Hz), showing contrast enhancement in the blood pool. Data was recorded at 4.7T on Bruker MR imager in a mouse sacrificed 1 minute after injection of Eu-polyLysine CEST (Example 4a) compound in water at a ˜0.6 mmol Eu(II)/kg dose (saturation pulse with a power of ˜25 μT for 2.0 s using 1000 Gauss pulses of 2 ms each).

Reference tubes contain from left to right: water, compound at 10 mM Eu(III) concentration, compound at 50 mM Eu(III) concentration and, Magnevist at a concentration such that T1˜1s.

A number of embodiments of the invention have been described. Nevertheless, it will be understood that various modifications may be made without departing from the spirit and scope of the invention. Accordingly, other embodiments are within the scope of the following claims.

What is claimed is:

1. A method for detecting a change in blood volume in one or more areas of a heart of a mammal, said method comprising:
a) administering a CEST-contrast agent to the mammal;
b) acquiring a first CEST image of the heart of the mammal in a rest state;
c) acquiring a second CEST image of the heart of the mammal in a stress state; and
d) comparing the two CEST images to evaluate blood volume changes in the one or more areas of the heart.

2. The method of claim 1, wherein said stress state in said mammal is induced via exercise.

3. The method of claim 1, wherein said stress state in said mammal is induced via a pharmacologic stressor.

4. A method for detecting a change in cerebral blood volume in one or more areas of the brain of a mammal, said method comprising:
a) administering a CEST-contrast agent to the mammal;
b) acquiring a first CEST image of the brain of the mammal in a rest state;
c) acquiring a second CEST image of the brain of the mammal in a stress state; and
d) comparing the two CEST images to evaluate blood volume changes in the one or more areas of the brain.

5. The method of claim 4, wherein said stress state in said mammal is induced via a visual stimulus, an olfactory stimulus, an auditory stimulus, a tactile stimulus, or a gustatory stimulus.

6. A CEST contrast agent or a pharmaceutically acceptable salt thereof having the structure: 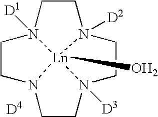
where:
D₁ is any of: 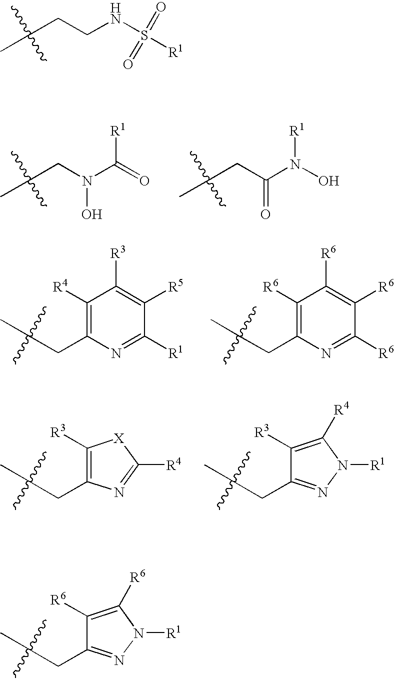
D², D³, D⁴ are any of 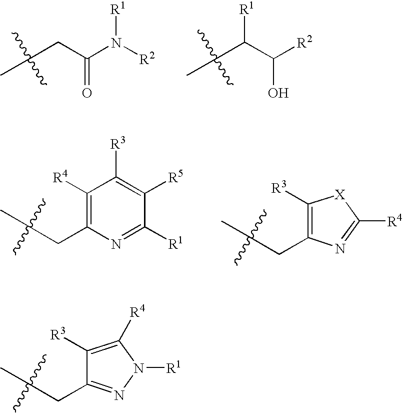
and
R₁=H, 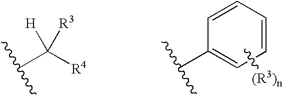
independently 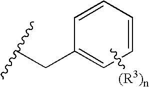
independently;
R²=H, 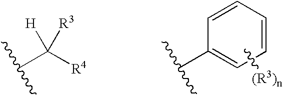
independently 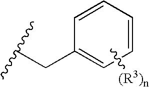
independently;
R³=H, alkyl, aryl, benzyl, halogen, carboxylate, sulfonate, or phosphonate;
R⁴=H, alkyl, aryl, benzyl, halogen, carboxylate, sulfonate, or phosphonate;
R⁵=H, alkyl, aryl, benzyl, halogen, carboxylate, sulfonate, or phosphonate;
R⁶=H, R³, R⁴, or together with the atoms to which it is attached forms a substituted or unsubstituted 5 or 6 member aromatic ring or heteroaromatic ring;
X=O, S, N—R¹;
Ln=Eu(III), Nd(III), Pr(III), Ce(III), or Yb(III); and
n=1-5.

7. A CEST contrast agent or pharmaceutically acceptable salt thereof having the structure: 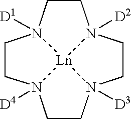
where Ln is Dy(III), Tm(III), Ho(III), Tb(III), Er(III), or Yb(III);
where at least one D¹, D², D³, D₄ is 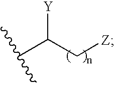
where n=0-4 and Z can be 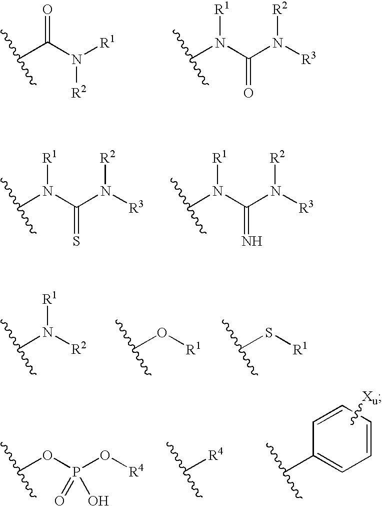
where R¹, R² and R³ are independently H; C((CH₂)^{s}—X)^{t} where t=1-3, s=1-6, and X=NH², OH, SH, CONH₂,NH—CO—NH₂, NH—C(NH)—NH₂, NH—NH₂, aryl-OH, aryl-SH; a sugar residue; CH₂-arylX^{u} where X is defined as above and u=1-5; and aryl-X^{u} where X and u are defined as above;
where R⁴ is defined as R¹ but can not be a hydrogen;
where Y is carboxylate, substituted or unsubstituted carboxamide, phosphonate, phosphonate ester, phosphinate, or phosphinate ester;
and the other D¹, D², D³, D⁴ are y 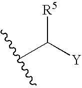
where R⁵ is H, alkyl, cycloalkyl, aryl, benzyl;
and Y is carboxylate, substituted or unsubstituted carboxamide, phosphonate, phosphonate ester, phosphinate, or phosphinate ester.

8. A CEST contrast agent or pharmaceutically acceptable salt thereof having the structure: 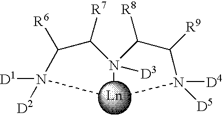
where Ln is Dy(III), Tm(III), Ho(III), Tb(III), Er(III), or Yb(III);
where at least one D¹⁻⁵ is 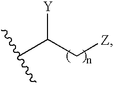
n=0-4 and Z can be 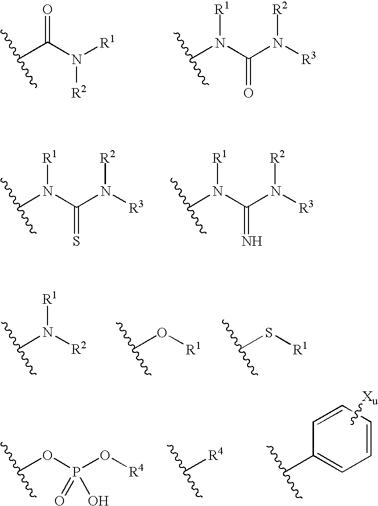
where R¹, R² and R³ are independently H; C((CH₂)^{s—}X)^{t} where t=1-3, s=1-6, and X=NH², OH, SH, CONH₂,NH—CO—NH₂, NH—C(NH)—NH₂, NH—NH₂, aryl-OH, aryl-SH; a sugar residue; CH₂-arylX^{u} where X is defined as above and u=1-5; aryl-X^{u} where X and u are defined as above;
where R⁴ is defined as R¹ but can not be a hydrogen;
where Y is carboxylate, substituted or unsubstituted carboxamide, phosphonate, phosphonate ester, phosphinate, or phosphinate ester;
where R⁶, R⁷, R⁸, and R⁹ are defined as R¹ and can also be (CH2)^{m}-Z
where m=0-6 and Z is defined above;
and the other D¹-⁵ are 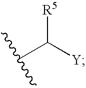
where R⁵ is H, alkyl, cycloalkyl, aryl, benzyl and Y is carboxylate, substituted or unsubstituted carboxamide, phosphonate, phosphonate ester, phosphinate, or phosphinate ester.

9. A CEST composition of matter having the structure:
(C-L)^{s}-P-(L′-E)^{t}
where C is a CEST contrast agent according to claim 7 or 8;
where E is selected from NH2, OH, SH, CONH2,NH—CO—NH2, CO-NR1R2, NH—C(NH)—NH2, NH—NH2, aryl-OH, and aryl-SH;
where R1 and R2 are as defined in claim 8;
where L and L′ are linkers;
where s=1-100 and t=0-100; and
where P is a polymer or dendrimer.

10. The contrast agent of claim 9 wherein P is selected from: polylysine, PAMAM dendrimers, polyvinylacetic acid, polyacrylic acid, hyaluronic acid, glycosaminoglycans, and derivatized dextrans.
